(19) United States
(12) Patent Application Publication
(10) Pub. No.: US 2022/0330904 A1
(43) Pub. Date: Oct. 20, 2022

(54) WEARABLE NARCOSIS ALERT

(71) Applicants: Scott VanDerKarr, Dublin, OH (US); Lori D. Meyers, Columbus, OH (US); Christopher Yerington, Columbus, OH (US)

(72) Inventors: Scott VanDerKarr, Dublin, OH (US); Lori D. Meyers, Columbus, OH (US); Christopher Yerington, Columbus, OH (US)

(21) Appl. No.: 17/231,175

(22) Filed: Apr. 15, 2021

(51) Int. Cl. A61B 5/00; A61B 5/0205; A61B 5/024; A61B 5/08; A61B 5/11; A61B 5/1455; G08B 21/02

(52) U.S. Cl. CPC A61B 5/746; A61B 5/0205; A61B 5/02416; A61B 5/0816; A61B 5/1118; A61B 5/14551; A61B 5/1112; A61B 5/4845; G08B 21/02

(57) ABSTRACT

Apparatus worn by a person, methods, and support system for monitoring physiological parameters of the person, providing timely, actionable information to prevent death due to overdose, and, through social or professional influences, may cause life-saving changes in behavior. The apparatus sends alerts to the person via audible or tactile means, and to concerned others, that drug usage, drug overdose, or other critical medical condition has likely occurred. Alerts to concerned others include geographical information indicating where the person is, so that medical assistance personnel may find the person. Geographical information includes latitude, longitude, the translation of latitude and longitude into a street address, and elevation information indicating on which floor in a building the person is.

### CROSS-REFERENCE TO RELATED APPLICATIONS

This is a divisional application of U.S. patent application Ser. No. 16/120,360 filed on Sep. 3, 2018, titled WEARABLE NARCOSIS ALERT, and priority is claimed for U.S. Provisional application No. 62/553,962, filed Sep. 4, 2017 also titled WEARABLE NARCOSIS ALERT, each of which is incorporated by reference.

This application claims priority to the contents of which are incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF THE TECHNOLOGY

The subject technology is in the technical field of overdose protection, through monitoring physiological indicators that suggest that drug usage has taken place or that a user's medical condition is approaching critical stages.

### BACKGROUND OF THE TECHNOLOGY

A clear, well-documented, devastating affliction upon modern society is drug abuse. At its extreme, drug abuse often leads to drug overdose, because the drug user's ability to think rationally is severely hindered. And, the nature of drug abuse itself is that increasing quantities of drugs are required to attain the sense of normalcy and calm that the drug user seeks. At some tipping point, overdose is triggered, although the effects may be unnoticeable to the user for quite some time. And, it may not be noticeable at all to the user, as the particular drug further alters the user's ability to process reality.

A person in opioid overdose has certain readily observable indicators that foretell anoxic injury and approaching death. These may include unconsciousness and unresponsiveness. The person may appear to be awake, but unable to speak. The person may appear to be intoxicated, confused, delirious, or having mood swings. Opioids repress breathing; thus, respiration would be slower than normal, or may have stopped. The resulting loss of oxygen changes the skin color of a person: lighter complexions turn bluish purple, and darker complexions turn grayish or ashen. A variety of other manifestations may arise, including chocking; loss of body control; no movement; constipation; nausea; vomiting; cold, clammy skin; and other skin discoloration under the fingernails and lips. If the condition is not reversed, the heart slows and eventfully stops.

And yet, certain other physiological indicators, if timely measured and analyzed, can provide alerts of drug usage and of impending critical medical emergencies. Equipment capable of detecting these indicators is typically available in intensive care units (ICU), which are in turn staffed with anesthesiologists, perianethesia care unit (PACU) nurses, and ICU nurses who are capable of interpreting and acting upon the indicators. Lives are saved with the timely application of technology and professional knowledge.

A person with a drug abuse problem, of course, is unlikely to be in an ICU or close to medical professionals when using drugs. Such a person tends to be alone or with others involved in the same activity, hidden from parents, away from caring friends, and out of view of law enforcement personnel. Thus, the drug user, whose location is unknown, may not detect that overdose has occurred, and that death is approaching.

If a person has overdosed, he or she may be saved upon administration of appropriate doses of an opioid antagonist such as naloxone (generic version of the Narcan® product). Injections of naloxone reverse the effects of overdose if timely applied. The Narcan® product version of naloxone includes recorded, verbal instructions that tell the user precisely how to use the product.

However, how would we detect that a person, alone, hidden from view, and unaware of their own peril, has overdosed? How do we find the person in order to administer aid? Thousands of lives are lost each year due to drug overdose, with the victims being found alone, hidden away. Thousands of lives could be saved if the victim were found in time and aid administered.

### LIMITATIONS OF PRIOR ART

Insler et al., U.S. Patent Publication US20170172522 A1, (“Insler”) discloses a method of providing assistance to an individual suspected of having overdosed on opiates. However, Insler requires respiration rate, pulse rate, SpO2, and blood pressure, and an array of sensors in a wearable apparatus to provide that information. The method and apparatus provides for injection of one or more doses of an opioid antagonist upon detection of an overdose condition. Insler also discloses an entirely voluntary approach, where the individual has complete control. The wearer using the Insler method and apparatus may choose to wear the device or not, and may disable alarms and features that would send alerts wirelessly to others who could provide aid. Insler also teaches use of the method and apparatus as an aid for abuse, where the wearer may use opiates and rely on the method and device as a personal safety measure. Furthermore, Insler provides for the wearer to stop the opioid antagonist injection if the ensuing withdrawal effects are too uncomfortable. This, of course, relies on the wearer, under the influence of opiates, acting rationally.

Vetlz, in U.S. Publication 20170173262 A1 (“Veltz”) discloses an array of physiological sensors and actuators for comprehensive collection of physiological information of a wearer, to be relayed wirelessly via cell phone and other wireless means, for highly sophisticated analysis and treatment. Such analysis and treatment would be on par to what would be available to a patient in a formal medical care facility. The information conveyed is detailed for medical accuracy, and conveyed securely for medical and privacy reasons. The wearer here cannot be mobile, given the array of sensors and actuators contemplated under Insler. The wearer would be bedridden, although not necessarily in a formal medical care facility, with medical professionals monitoring for various indications.

### Need for Subject Technology

What is needed is a device and system worn by the drug user at all times, that cannot be readily removed by the drug user, and that monitors a minimal physiological parameters that indicate drug usage and overdose. No greater precision is needed. Such a device and system would send alerts both to the wearer via audible or vibration means, and to concerned third parties via wireless means that drug usage and drug overdose has likely occurred. Audible alerts have proved effective in causing a person under the influence of drugs to become aware of their peril, so that they may seek help before it is too late. Furthermore, such alert to concerned third parties would include geographical information indicating precisely where the wearer is, so that people providing medical assistance may find the wearer. The geographical information would include, for example, latitude, longitude, the translation of latitude and longitude into a street address, and, when possible, elevation information that might indicate on which floor in a building the drug user might be. This provides several opportunities to prevent death due to overdose. First of all, upon indication of drug use, someone may have time and information to find and to intervene before overdose. Secondly, if overdose occurs, aid can be dispatched before death claims the user.

A critical aspect of the technology is reliance on concerned third parties. These would include family and friends, whose interest is keeping the wearer alive and supporting treatment and recovery away from addiction. Law enforcement officials would have the same concerns in addition to and in cooperation with court ordered provisions. Panebianco et al. in Personal support networks, social capital, and risk of relapse among individuals treated for substance use issues (International Journal of Drug Policy, Volume 27, January 2016, Pages 146-153), is among the many literature references that show the value of a support network in substance abuse treatment. Further support is provided by Seyyedi et al., The Role of Social Networks in Prevention Drug Abuse In School, European Scientific Journal, May edition volume 8, No, 10), showing the value of peer group support. Lezin et al. in Parent-Child Connectedness, Implications for Research, Interventions, and Positive Impacts on Adolescent Health (ETR Associates, 2004, recapp.etr.org/recapp/documents/research/litreview.pdf) conclude,
“The researchers concluded that parental monitoring is indeed an effective tool in preventing and ameliorating drug use.”
   Further conclusion is that monitoring boys who were heavy drug users, to use less, and that girls tended to stop usage under parental monitoring.

### SUMMARY OF THE TECHNOLOGY

The subject technology comprises a wearable narcosis alert device and system that is worn about the wrist or elsewhere on the body, worn by the person who is at risk of overdose. Information about the location of the wearer would be provided by the global navigation system technology. Communication to deliver information to appropriately approved concerned third parties is provided through wireless or wired technologies.

Also provided on the wearable narcosis alert device and system would be means for delivering audible or tactile alerts to the wearer. One reason for that is that a person in or approaching narcosis may not be aware of the approaching danger. Audible and tactile alarms have proved to be effective in causing a person in such condition to take immediate measures to avoid overdose.

Sensors in the wearable narcosis alert device and system provide pulse oximetry data, using photoplethysmography technology. Available information from pulse oximetry include pulse rate as a measure of heart rate and peripheral capillary oxygen saturation (“SpO2”) as an approximation of arterial oxygen saturation (“SaO2”). Photoplethysmographic sensors are commonly available and in use in a variety of fitness, wrist-worn devices, such as the FitBit® and Garmin fitness tracking devices.

The information, properly processed and recorded, would be suitably reliable and accurate for a computer system, whether embedded in the device, external to it, or a combination of embedded and external, to make deductions concerning the wearer's medical condition, and to issue alerts about the wearer's condition and location.

Respiration rate is an important parameter in diagnosing narcosis. Respiration rate is not directly available from photoplethysmographic pulse rate information, but can be derived from that information with sufficient accuracy to indicate drug use, drug overdose, or other medical condition.

Another important parameter is body activity, or more generally, movement. Individuals who are under the influence of narcotics generally have less movement than individuals who are not under the influence. Movement alone is not a sufficient indicator of narcotic use. However, movement information when analyzed in combination with other information has probative qualities. Fitness, wrist-worn devices, such as the previously cited FitBit® and Garmin fitness tracking devices typically also include accelerometers capability which can provide movement information.

Thus the information required to make the determinations contemplated here are:
heart rate
SpO2 as an approximation of SaO2
respiration rate as estimated from photoplethysmography data
movement as estimated from accelerometer information

Fitness, wrist-worn devices, such as the previously cited FitBit® and Garmin fitness tracking devices typically also include wireless communication capability which can convey physiological information to others as well as audible and vibration alerts. Furthermore such fitness devices can provide the information required to make a determination of drug usage and drug overdose. However, the proposed wearable narcosis alert device and system as described herein is novel, and uniquely provides and applies the analysis required to determine drug use and overdose.

### BRIEF DESCRIPTION OF THE DRAWINGS

FIG. 1A shows a typical image of heart electrical activity, as shown in an electrocardiogram tracing.

FIG. 1B denotes various peaks and valleys of interest in the electrocardiogram tracing.

FIG. 1C shows a typical image of heart mechanical activity as represented by pulse rate, as shown in a photoplethysmography tracing.

FIG. 2 is a perspective view of the wearable narcosis alert device as worn.

FIG. 3 is a system level architecture diagram of the wearable narcosis alert device.

FIG. 4 is a process flow operating sequence diagram of the wearable narcosis alert device.

FIG. 5 is a decision table showing criteria used to determine medical conditions for which alerts would be issued.

FIG. 6A shows heart rate variability as applied to electrocardiogram tracings. FIG. 6B shows heart rate variability as applied to pulse rate in PPG tracings.

FIG. 7 shows heart rate and respiratory rate correlation.

FIG. 8 depicts respiratory rate spectral density.

### DETAILED DESCRIPTION

The subject technology will be described more fully with reference to the accompanying drawings, in which a preferred embodiment of the subject technology is shown. However, persons of ordinary skill in the appropriate arts may modify the subject technology described here while still achieving the favorable results. Accordingly, the description which follows is to be understood as being a broad, teaching disclosure directed to persons of ordinary skill in the appropriate arts, and not as limiting upon the subject technology.

Certain definitions are stated to assist in interpreting this description and the Figures.

By “heart” we refer specifically to the human heart and its associated anatomy, physiology, and properties.

“Narcosis” is a state of stupor, unconsciousness, or arrested activity produced by the influence of narcotics or other chemicals or physical agents.

A “narcotic” is a drug that in moderate doses dulls the senses, relieves pain, and induces profound sleep but in excessive doses causes stupor, coma, or convulsions. Alternatively, a narcotic is a drug that is subject to restriction by law similar to restrictions placed upon addictive narcotics, regardless whether in fact the particular drug is physiologically addictive.

An “opiate” is a drug containing opium, or derived from opium, and tending to induce sleep and to alleviate pain.

An “opioid” is a drug that possesses some properties characteristic of opiate narcotics, and includes heroin, synthetic variants such as fentanyl, and prescription pain relievers (such as OxyContin®, Vicodin® (hydrocodone), codeine, morphine, and similar drugs).

An “overdose,” as a noun, comprises a dose of a drug or other agent that is greater than one that is safe for the particular person, resulting in toxic or lethal consequences. Alternatively, the verb “to overdose” means to give an overdose or too many doses to a person, or to take or experience an overdose (as in having “overdosed on” an opiate).

“Anoxia” means a deficiency of oxygen reaching the tissues of the body, especially of such severity as to result in permanent damage.

An “electrocardiogram” (interchangeably referred to as “ECG” and “EKG”) is a device that renders a graphical representations (“tracings”) of heart mechanical activity over time, including heart rate, as indicated by the heart's electrical activity.

“Heart rate” means the sequence and pattern of heart beats over time, as represented by pulse or electrical indications.

“Photoplethysmography” (“PPG”) is a non-invasive technology that derives information about volumetric changes in blood and blood flow. Light at specific frequencies and intensities is applied to a person's blood vessels. Analysis of absorption of that light, either by spectral examination of the reflections of that light or spectral examination of the light that passes through those blood vessels, provides the information. Information derived through PPG analysis include, with varying accuracy:
blood oxygen saturation
heart rate
blood pressure
heart rate variability
respiration volume and rate

“Heart Rate Variability” (“HRV”) concerns the variation in time interval between consecutive heart beats, as measured between amplitudes of successive R intervals. Other terms for HRV include “cycle length variability,” “RR variability,” and “heart rate variability.” Research has shown, and is well-accepted in the medical community, that the interval between heart beats varies from heart beat to heart beat over time for a variety of causes; and that analysis of HRV is revealing of other physiological information. In addition to medical and physical fitness uses, HRV is also being used as biofeedback for purposes of reducing stress, promoting relaxation, and promoting other body autonomic activity such as meditation. For our purposes, HRV analysis can reveal respiration rate, which is a critical indicator of narcosis.

“Global navigational system” refers to methods and devices for revealing precise physical location on Earth by latitude and longitude or other measures. Typical global navigational systems include “Global Positioning System” “(GPS,” United States), Giobalnaya navigatsionnaya sputnikovaya sistema (“GLONASS” of the Russian Federal Space Gancy), Galilaeo, Beidou, The Indian Regional Navigation Satellite System (“IRNSS”), the Quasi-Zenith Satellite System (“QZNSS”, India), and the like.

“Wireless technology” includes far field technologies such as cellular, WiFi®, and similar technologies; and near field technologies such as Bluetooth®, Zigbee®, near field magnetic, and similar technologies.

“Tactile” indication is one that is felt through sense of touch, such as from a vibration device on the wrist, ankle, or other part of the body.

The subject technology is founded, in part, the following premises:
1) If the overdosed person is found and appropriate aid is delivered before death, the person may survive.
2) Overdose can be determined from observation of the person's respiration rate, heart rate, blood oxygen level, and quality and quantity of movement. The observation looks for deviation from norms, where the norms have been established over at least one circadian period and are updated periodically. The observation entails looking back to previous updated norms at various intervals in order to decide whether an abnormal event has occurred.
3) The nature and level of movement of the person can provide supporting evidence of narcosis, overdose, or other serious medical condition.
4) We can measure heart rate (“HR”) and oxygen level (O2 and/or SpO2) at the wearer's wrist, from readily available photoplethysmographic (“PPG”) sensors.
5) Respiration rate can be sufficiently estimated from analysis of heart rate data, through analysis of heart rate variability (“HRV”) in particular. The result is sufficient to make decisions about whether the person is in narcosis or has overdosed.
6) Readily available accelerometer sensor devices can provide information about the nature and level of the person's movement.
7) Global navigation system technology can establish location of the person, with reasonable accuracy.
8) Upon determination that a person is in narcosis, has overdosed, or is in peril from other medical conditions, information about the person's condition and location may be conveyed to interested or concerned third parties.
9) Wireless communication means can convey that information to other interested parties.
10) Audible or tactile alarms may be triggered in effort to alert the person of the danger, so that possibly self-help may be initiated (such as issuing a call for help or moving to a more visible location).
11) A wearable device may operate on or effect the premises above to save lives.

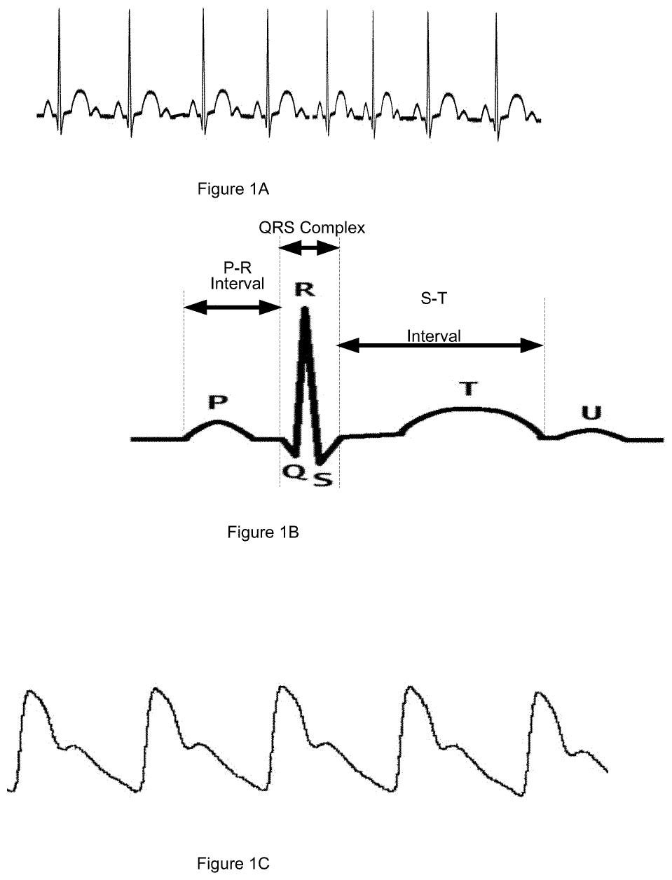

The familiar graphical representation of the beating heart as shown in an ECG tracing is shown in FIG. 1A, representing two successive heart beats as successive positive and negative humps, or peaks and valleys.

Referring now to FIG. 1B, in the first half of the hump marked with the letter P (also called the “P-wave”, the atria fill, passively, with blood. During the second half, from P to just before the hump marked with the letter Q, the atria contract. During the period after the crest of the P-wave and indicated by the hump marked Q, all heart valves close. Then, from the end of the Q hump and through the R hump, pressure builds in the ventricle chambers. During the S hump and through the first half of the T hump, the semilunar valves open and the ventricles eject blood. During the next part of the T hump, the ventricles relax and all valves close, before the start of the next cycle.

FIG. 1B further depicts what is commonly called the “QRS complex” (or, the “QRS Interval”), comprises the adjacent and sequential intervals Q, R, and S. It is the most prominent part of an ECG tracing, and represents depolarization of the left and right ventricles of the heart. Indeed, the amplitude of the R component is quite visually distinctive in the ECG tracing. Depolarization represents the leading edge of the R interval, causes contraction in the left and right atria of the heart during the R interval, and depolarization during the trailing edge of the R interval. In layman's terms, a heart beat is represented by the sequential intervals Q, R, S, and T.

FIG. 1C shows a typical image of heart mechanical activity as represented by pulse rate, as shown in a PPG tracing. The adjacent high and low peaks represent one pulse event, and thus one heart beat. The high peak lags the QRS complex in time, as the PPG signal represents the result of heart contractions triggered due to the QRS Complex. And yet, a one-to-one correspondence exists between each QRS complex event and each pulse.

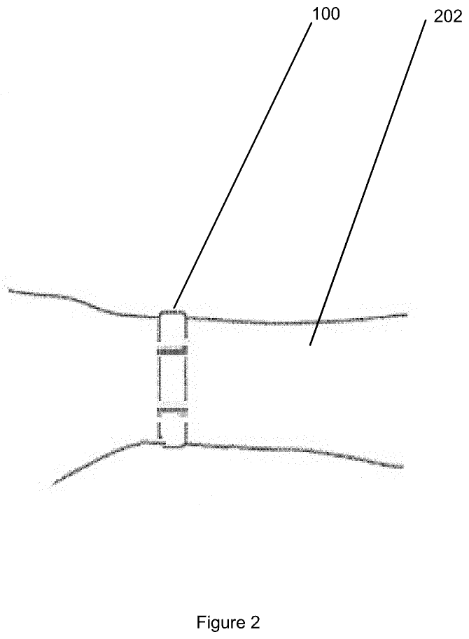

FIG. 2 shows the wearable narcosis alert device 200 as worn, in one embodiment, on the wearer's wrist 202. In other embodiments, the wireless narcosis alert 200 would be worn on an ankle or other body part suitable for the sensors contemplated here.

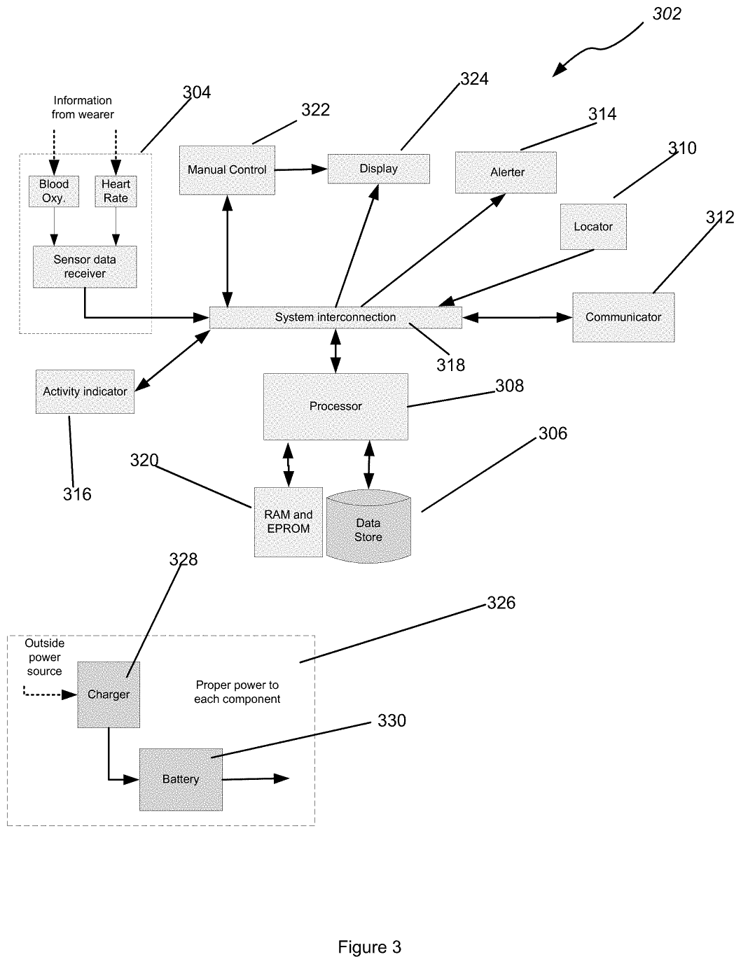

FIG. 3 is a system level architecture 302 of the wireless narcosis alert 200. It shows various hardware components required to achieve objectives. Connection through the outside world would be by one or more wireless technologies. Primary components include sensors 304, a processor 308, a data store 306, a program store 320, a locator 310, a communicator 312, an alerter 314, and an activity indicator 316. System interconnection 318 provides appropriate electrical and signal connectivity among all components. A power subsystem 326, further comprising a charger 328 and a battery 330, supplies electrical energy to all components. The person wearing the wireless narcosis alert 200 would have access to a display 324 showing useful information. Manual control 322 provides limited capabilities for the wearer to operate the wireless narcosis alert 200, such as selecting operating modes, selecting information to be displayed, alarm volumes, and other parameters as allowed.

In one embodiment, sensors 304 provide pulse oximetry data, which includes heart and SpO2. Heart rate variability and blood oxygen level would be deduced from that data, as would respiration rate. The locator 310, typically using global navigational system technology provides information about the wearer's location. The activity indicator 316, typically including accelerometer technology, provides information used to interpret the wearer's movement or non-movement.

The processor 308 manages all tasks, including collecting and processing data, directing communication with the outside world, decision-making based on data, and issuing alerts and alarms. The processor 308 may be an array of task-oriented processor subsets, where a subset is tasked with acquiring information from sensors 304; another subset is tasked with processing, organizing, and storing data; another subset tasked with analysis of data; another subset tasked with establishing and maintaining communication; and another subset tasked with alerts. The wireless narcosis alert 200 must have reliable and sufficient power from the power subsystem 326. The battery 330 would be charged via charger 328 from conventional wall outlets, or from alternative sources such as solar or motion-based technology. The program store is provided to hold configuration information and programs for program execution. The data store 306 is provided to hold baseline and historical data used in analysis of the wearer's medical condition.

The display 324 provides visual information to the wearer. Manual control 322 is provided to the wearer for such operations as selecting modes, selecting information to be displayed, alarm volumes, and other parameters as allowed.

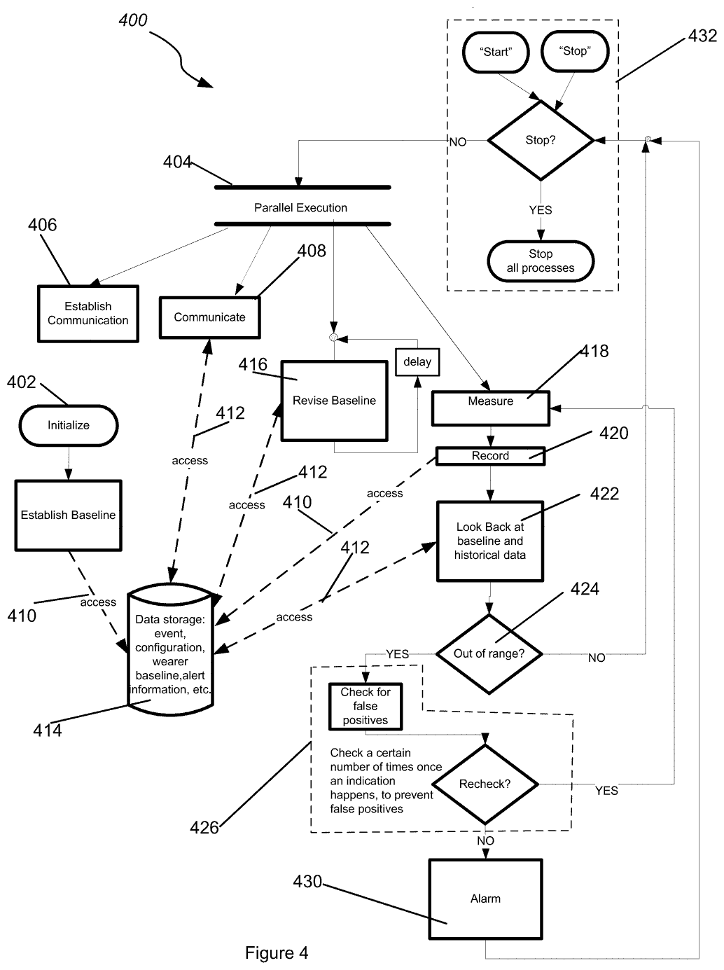

FIG. 4 is a process flow diagram 400 showing general operation, and comprises critical analyses steps and decisions made within the wireless narcosis alert 200. Several processes run in parallel. These include generally, but are not limited to:
1) Establishing and maintaining wireless contact with the outside world
2) Periodic off loading (or up loading) data via wireless technology
3) Establishing and maintaining baseline data about the particular wearer
4) Primary loop for updating, checking, and interpreting sensor data, and alarming appropriately when needed based on physiological conditions detected.

As shown in FIG. 4, the wireless narcosis alert 200 may undergo an initialize 402 step prior to being provided to the wearer, including establishing initial baseline information for the wearer, such as wearer identification, weight, height, age, and initial PPG data. That initial baseline information is stored via write access 410 into a database 414, and is further updated throughout operation of the WNA.

When the wireless narcosis alert 200 is turned on and off via manual control 432, that operation initiates the parallel execution 404 of the following execution branches: establish communication. 406; communicate 408; revise baseline 416; and the sequence comprising measure 418, record 420, look back 422, and analysis of data to determine whether an alarm 430 step must be executed.

Establish communication 406 entails making and maintaining contact with the outside world, generally via wireless technology, so that alert and location information may be delivered in timely manner. Communicate 408 occurs as needed to send alert and location information at appropriate times, and to respond to request for information from the outside world. Revise baseline 416 comprises the periodic correction of the wearer's PPG information as collected in database 414, to establish norms from which out-of-range determinations are made. The execution branch that begins with measure 418 is a loop that runs until stopped via manual control, and includes measure 418 current wearer data; record 420 current wearer data (via write access 410) into database 414; a look back 422 at data in the database 414 (via read/write access 412) at various time intervals; and analysis of current data for the wearer compared with historical and baseline data. A check for out of range 424 in any of the monitored parameters invokes a false positive check 426, in effort to avoid false alarms. If the false positive check 426 indicates that the data are correct, an alarm 430 sequence is initiated. The alarm 430 sequence invokes the communicate 408 step to issue appropriate audible and tactile alerts to the wearer as well as issue appropriate alerts to the outside word.

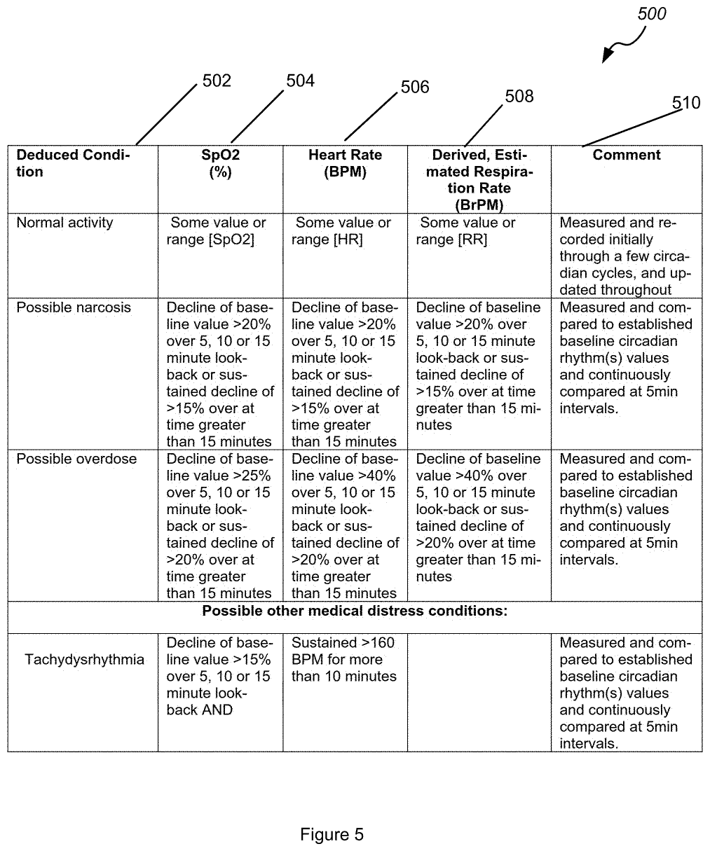

FIG. 5 comprises a table 500 of detail of the primary decision-making criteria for alerting on medical conditions. The table 500 reflects the analysis performed by the WNA, as directed by the flow diagram 400 and executed through the system architecture 302. For each particular wearer of the wearable narcosis alert device, recorded information provides the individual's baseline establishing “normal activity.” The baseline would be continually updated throughout the period that the individual wears the WNA. Variation from this baseline, observed in real time and compared with recent history, would indicate certain conditions upon which the device would provide alerts to interested parties. Information categories in the table 500 include deduced condition 520, which comprises the suspected condition of the wearer based on analysis; SpO2 504 as derived from wearer PPG data; heart rate 506 as derived from wearer PPG data; and respiration rate 508 as computed from wearer heart rate.

One approach and embodiment includes:
i) Measure and record pulse oximetry parameters over at least one circadian cycle to establish an initial baseline.
ii) Measure periodically thereafter and update baseline
iii) With each measurement, look back at data from each of the following:
   24 hours ago (Circadian comparison),
   4 hours ago,
   2 hours ago,
   1 hour ago,
   30 minutes ago,
   5 minutes ago
iv) Certain variations from the baseline or patterns from analysis of the look back provide evidence from which deduction of a condition can be made, with a certain acceptable probability level.

The deduced conditions 502 and associated heart rate 506 and SpO2 504 in FIG. 5 table 500 are well-known among anesthesiologists, perianesthesia (PACU) nurses, and intensive care unit (ICU) nurses. However, the wireless narcosis alert 200 organizes and analyzes available information much quicker than would a human medical professional could, and provides information organized to interested parties in remote locations, and provides alerts to the affected person, and provides alerts to concerned third parties. The result is actionable information is provided in timely fashion to prevent death due to overdose, through social or professional influences, may cause life-saving changes in behavior.

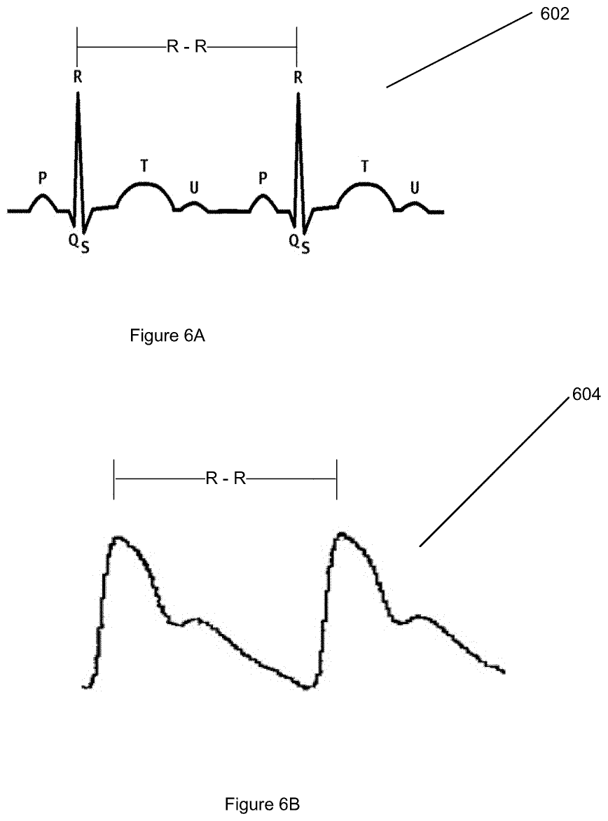

FIG. 6A depicts heart rate and heart rate variability 602 in an EKG tracing. FIG. 6B depicts heart rate and heart rate variability 602 in a PPG tracing. The time between R and R is the interval between heart beats. That time is not uniform, and changes throughout the day and night for a variety of physiological reasons. Heart rate variability is critical in determining respiration rate. There are several ways to derive respiration rate form pulse oximetry data. One way is through an algorithm called RRoxi, an algorithm for pulse oximetry derived respiration rate (“Developing an algorithm for pulse oximetry derived respiration rate (RRoxi): a healthy volunteer study,” Addison, Watson, Mastek, Mecca, J Clin Monit Comput (2012), 26:45-51, DOI 10.1007/s10877-011-9332-y, hereinafter the “RRoxi 1 study”). This method recognizes that the PPG signal is modulated by 3 parameters. A baseline modulation, or “baseline wander,” (termed “DC”, as opposed to the alternating, or “AC”, nature of the primary PPG signal) parameter exists. When a person breaths in, small decreases in central venous pressure result in greater blood flow in the veins. When a person breaths out, blood flow is shunted, and a sequence of filling and draining adds modulation to the baseline. Thus, base line modulation is an indicator of respiration. Pulse amplitude modulation, also called simply “amplitude modulation,” reflects decreased left ventricle stroke volume during inspiration. This leads to decreased pulse amplitude during inspiration, an thus is an indicator of inspiration. Finally, the combination of RSA and HRV is a modulation, where the time between heart beats varies due to respiration (among other influences): longer during expiration and shorter during inspiration. HRV, in essence, is “frequency modulation.” The RRoxi algorithm processes the three modulations using continuous wavelet transform analysis methods and a particular wavelet transform, applied every 5 seconds from a previous 45 seconds data sample, to derive respiration rate at high correlation to actual, measured respiration rate. Further studies of the RRoxi algorithm applied to persons with various medical conditions confirmed the accuracy of the algorithm. (“Pulse oximtery-derived respiration rate in general care flow patients,” Addison, Watson, Mestek, Ochs, Uribe, Bergese, J Clin Monit Comput (2015) 29:113-120, DOI 10.1007/s10877-014-9575-5, hereinafter “RRoxi 2 study”).

Another way to derive respiratory rate from pulse oximetry data concerns HRV and respiratory cardiac variance analyzed via several alternative time domain and frequency domain methods. Again, it is well-known that heart rate increases and the interval between heart beats is shorter when a person inspires. Additionally, the opposite is well-known: that heart rate decreases and the interval between heart beats is longer when a person expires. Time domain and frequency domain methods described in detail in “Heart rate variability, Standards of measurement, physiological, interpretation, and clinical use”, Europeans Heart Journal (1996) 17, 354-381, hereinafter the “HRV study,” show how respiration rate is derived from this information. Again, correlation between derived and actual respiration rate is high.

Yet another method uses HRV and measurement of cardiorespiratory coordination with a “phase coordination ratio.” (Edelhauser, Hak, Kleinrath, Luhr, Matthiessen, Weinzirl, and Cysarz, “Impact of colored light on cardiorespiratory coordination,” Evidence-based Complementary and Alternative Medicine, December 2013, DOI: 10.1155/2013/810876 Source PubMed). Again, relying on HRV and changes in the interval between heart beats due to inspiration and expiration, this method tracks the changes over time to mark respiration cycles. The method creates a “heart rate respiration ratio,” a “phase coordination ratio,” and applies weighted averages at 5 minute epochs to compute “heart beats per respiratory cycle.” The result is the filtering out non-respiratory causes of HRV, leaving an accurate estimation of respiratory rate.

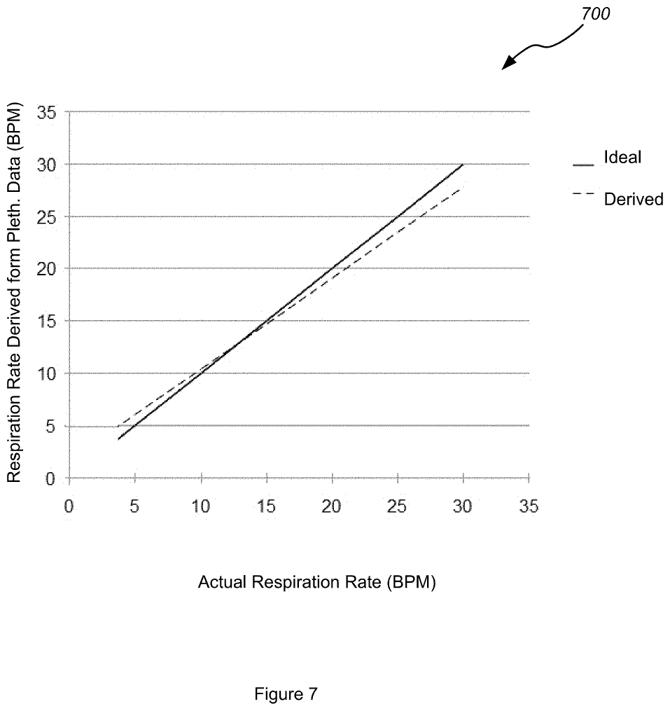

FIG. 7 depicts the correlation 700 between heart rate and respiration rate, from the RRoxi 1 study. An ideal would be an exact match between actual respiratory rate and the rate derived from PPG data. Experimental data from the RRoxi 1 study was collected, and then a best fit algorithm applied, to reveal the correlation shown in FIG. 7.

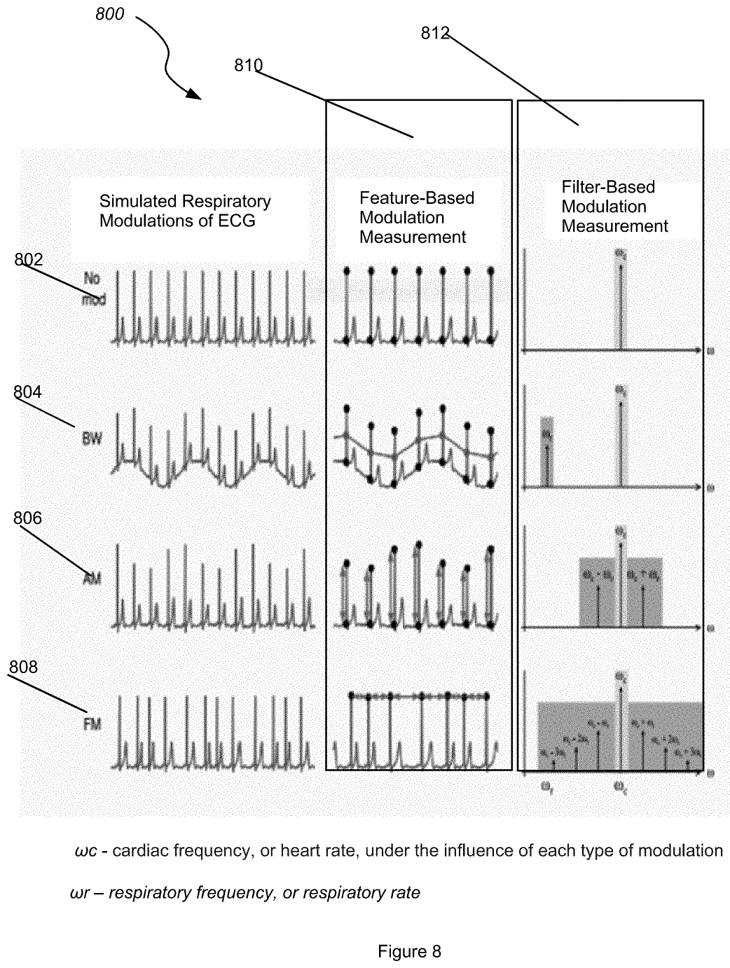

FIG. 8 is depicts table 800 derived from “Extraction of respiratory signals from the electrocardiogram and photoplethysmogram: technical and physiological determinants.” Alastruey, et al.; Physiol. Meas. 38 (2017) 669-690 (hereinafter the “Extraction study”). It shows ECG tracings as modulated by respiration rate, and detection of respiration rate from analysis. Shown are a non modulation trace 802, a base wander trace 804, an amplitude modulation trace 806, and a frequency modulation trace 808. For each, frequency spectral analysis was conducted, with the results as shown for cardiac frequency and respiratory frequency. The results show that only the base wander modulation is manifested in the cardiac frequency band.

### BEST MODE OF THE PREFERRED EMBODIMENT

A preferred embodiment of the wearable narcosis alert device entails its uses in alerting third parties when the wearer is under narcosis or other medical distress, to provide location information so that medical aid may be dispatched, and to provide an alert to the person that dangerous levels of narcosis may be approaching. The system serves at least three potential needs and alternative embodiments:
1) Court-ordered enforcement, with appropriate law enforcement officials solely empowered to remove the wearable narcosis alert device. The wearer must volunteer initially to wear the wearable narcosis alert device.
2) Remote patient monitoring by care providers, with less severe restrictions on removal.
3) Remote monitoring by concerned third parties, such as family and friends, with some restrictions on removal, at least preventing removal by the wearer. The wearer must volunteer initially to wear the wearable narcosis alert device, or be compelled by a parent with legal capacity to do so.

As additional information may be available with improvements in sensor technology applied to the wearable narcosis alert device as worn on the wrist, ankle, or other single body location, additional deductions may be made and alerts generated.

Data collected from the wearer may be made available, in appropriate and law-compliant manner, to the particular person's medical records provided to heath care providers, as well as to researchers.

Also, the system should also react to some on-demand polling, instead of waiting for the periodic upload. And, the system should allow on-line software updates.

While the foregoing written description enables one of ordinary skill to make and use what is considered presently to be the best mode thereof, those of ordinary skill will understand and appreciate the existence of variations, combinations, and equivalents of the specific embodiment, method, and examples herein. Unless claimed, particular system architecture and algorithms shown are not critical, but represent one or more embodiments. The wearable narcosis alert technology presented here should therefore not be limited by the above described embodiments, methods, or examples, but by all embodiments and methods within the scope and spirit of the subject technology.

We claim:

1. A method for issuing an alert upon evidence of one or more medical conditions, comprising:
establishing a baseline of values for a set of one or more physiological parameters for a person;
said establishing being derived from measurement over at least one Circadian period;
the baseline further establishing in-range and out-of-range conditions for each such physiological parameter;
periodic measurement of the set of physiological parameters at pre-determined intervals;
periodic recording of the set of physiological parameters at the pre-determined intervals;
periodic detecting and recording the geophysical location of the person;
revising the baseline based upon the periodic recording of the set of physiological parameters;
performing a series of look backs at previously recorded physical parameters upon one or more periodic measurements being out of range;
analyzing the series of look backs according to a set of prescribed patterns for detecting the one or more medical conditions;
communicating an alert upon detecting the existence of the one or more medical conditions and the geophysical location of the person;
wherein the alert is communicated to the person and to one or more other persons;
wherein the one or more other persons comprise one or more networks of persons acting in private or professional capacity and who have a right and need to receive the alert;
and wherein the person is prevented from interfering with the method.

2. The method of claim 1 wherein the set of one or more physiological parameters comprises heart rate, blood oxygen level, respiration rate derived from heart rate, and activity.

3. The method of any of claim 1 wherein the one or more medical conditions comprises use of narcotics, wherein the use ranges from no use to overdose.

4. The method of any of claim 1 wherein the one or more medical conditions comprises tachy-dysrhythmia.

5. The method of any of claim 1 wherein the one or more physiological parameters is derived from photoplethysmography.

6. A health support system for an addicted person comprising:
a set of other persons having a legal right and need to know geophysical location and medical condition of the addicted person;
a computer system configured for forming an analysis based upon one of more physiological parameters of the addicted person, wherein the one or more physiological parameters is measure periodically;
a locator configured for determining the geophysical location of the person;
an alerter configured for alerting the set of other persons of results of the analysis,
wherein the analysis further comprises
establishing a baseline of values for the set of one or more physiological parameters for a person,
said establishing being derived from measurement over at least one Circadian period;
the baseline further establishing in-range and out-of-range conditions for each physiological parameter;
periodic measurement of the set of physiological parameters at intervals;
periodic recording of the set of physiological parameters at the intervals;
periodic detecting and recording the geophysical location of the person;
adjusting the baseline based upon the periodic recording of the set of physiological parameters;
upon one or more periodic measurements being out of range, perform a series of look backs at previously recorded physical parameters;
a communicator for conveying location information of the person to the other persons;
and a preventer configured for preventing the person from interfering with the conveying of the geophysical information to the other persons and preventing interfering with the alerting.

7. The health support system of claim 6 wherein the other persons comprise a set of one or private persons.

8. The health support system of claim 6 wherein the other persons comprise a set of persons empowered by courts of appropriate jurisdiction to monitor the activities of the person.
